(19) United States
(12) Patent Application Publication
(10) Pub. No.: US 2003/0165557 A1
(43) Pub. Date: Sep. 4, 2003

(54) Food, medicine and method relating to Bifidobacterium proliferation promoting action, allergy preventive action and human cholesterol lowering action

(75) Inventors: Hirohisa Suido, Osaka (JP); Ako Ikeda, Osaka (JP); Toshio Tabei, Osaka (JP); Mayumi Kotani, Kobe-shi (JP); Akihito Fujita, Osaka (JP); Akira Takeuchi, Osaka (JP)

Correspondence Address: SUGHRUE MION, PLLC, 2100 Pennsylvania Avenue, NW, Washington, DC, 20037-3213, US

(73) Assignee: SUNSTAR, INC.

(21) Appl. No.: 10/373,106

(22) Filed: Feb. 26, 2003

Related U.S. Application Data
(62) Division of application No. 09/646,540, filed on Sep. 19, 2000, now abandoned.
Application No. 09/646,540 is a 371 of international application No. PCT/JP99/01354, filed on Sep. 18, 1999.

(51) Int. Cl. A61K 47/00; A61K 35/78; A23L 1/00

(52) U.S. Cl. 424/439; 424/755; 424/745; 426/615

(57) ABSTRACT

A food, medicine and cosmetic containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R. Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves; and a method for preventing or treating a disease, comprising ingestion or administration of an effective amount of these vegetables and fruit.

### TECHNICAL FIELD

The present invention relates to foods and medicines containing broccoli, shepherd's purse and like plants; and methods for preventing or treating a disease using broccoli, shepherd's purse and like plants.

### BACKGROUND ART

Recent studies have revealed that the intestinal flora has various functions such as formation or inactivation of carcinogenic substances or senility accelerating substances, and activation or toxification of drugs, in the intestines. In the intestinal flora, Bifidobacteria are known to have functions such as: protection of the body from enteric infection and food poisoning caused by pathogenic bacteria; suppression of intestinal putrefaction to thereby reduce carcinogenesis-related enzymatic activities and formation of putrefaction products; prevention of constipation by promotion of intestinal motility; improvement of the immuno function of the host by stimulation; decomposition or adsorption of carcinogenic substances; and production of Vitamin B group (J. Antibact. Antifung. Agents Vol.25, No.4, pp 219-231, 1997). Attempts have been made to maintain predominance of Bifidobacteria having these functions in the intestines, by administering to humans Bifidobacterium proliferation promoting materials by themselves, or foods or drugs containing such materials.

Known Bifidobacterium proliferation promoting materials include saccharides such as N-acetylglucosamine, lactulose, raffinose, stachyose, maltotriose (“Bifidobacteria”, p 77, 1979, Yakult Honsha Co., Ltd.), fructooligosaccharide (“Kagaku to Seibutsu (Chemistry and Organism”, Vol.21, p. 291, 1983, Gakkai Shuppan Center), galactooligosaccharide (Japanese Examined Patent Publications Nos. 1983-20266 and 1986-46479 and Japanese Unexamined Patent Publication No. 1985-41449), isomaltooligosaccharide (Japan Nutritional Science Society, 1986), theanderose (Japanese Unexamined Patent Publication No. 1991-183454), cyclodextrin (Japanese Unexamined Patent Publication No. 1982-138385), and konjac mannan (“Riken Intestinal Flora Symposium, Intestinal Flora and Nutrition”, p. 89, 1983, Gakkai Shuppan Center); carrot extract (comprising pantetheine as the main component; “Bifidobacteria”, p. 77, 1979, Yakult Honsha Co., Ltd.); soy milk (Japanese Examined Patent Publication No. 1970-9822); soy milk extract (Japanese Unexamined Patent Publication No. 1984-17906); extract of nonpathogenic Escherichia coli culture solution (Japanese Examined Patent Publication No. 1975-13359); an enzymatic decomposition product of barley protein-containing material (Japanese Unexamined Patent Publication No. 1986-282070); calabash gourd powder (Japanese Unexamined Patent Publication No. 1988-291579); alcohol extract of residue after extraction of oil soluble components of soybeans (Japanese Unexamined Patent Publications Nos. 1987-155082 and 1985-66978); syrup extracted from calabash gourd fruit (Japanese Unexamined Patent Publication No. 1990-135088); tea extract (Japanese Unexamined Patent Publication No. 1989-191680); Araliaceae family plant extract (Japanese Unexamined Patent Publication No. 1990-249482); a dry potato product (Japanese Unexamined Patent Publication No. 1994-217733); extract of Coffea L. leaves (Japanese Unexamined Patent Publication No. 1994-125771); Chinese cabbage (Japanese Unexamined Patent Publication No. 1990-242667); and citrus fruit (Japanese Unexamined Patent Publication No. 1990-273155).

Many of the known Bifidobacterium proliferation promoting materials show a low effect in practical use, or have a low selectivity to enterobacteria so that they are utilized by harmful bacteria other than Bifidobacteria to thereby produce toxic gases. Further, some of the known materials necessitate complicated manipulation for production, or are expensive. Therefore, the known Bifidobacterium proliferation promoting materials are not satisfactory in all respects.

Accordingly, a Bifidobacterium proliferation promoting material is desired which has a high Bifidobacterium proliferation promoting activity and can be produced with ease at low cost. Such a material, if having a high safety in respect of food hygiene, would be useful not only as an additive for culture media, but also as a drug for controlling intestinal function or as a health oriented functional food material, hence having an extremely high industrial value.

Further, various allergies have become problems recently, and therefore inexpensive, highly safe, pharmaceutical compositions for preventing or treating allergies, and foods for preventing allergies are desired.

Moreover, in recent years, an increased number of middle to senior aged people and children suffer from lifestyle-induced diseases such as myocardial infarction and arteriosclerosis. One of the main risk factors of myocardial infarction and arteriosclerosis is hypercholesterolemia.

Hypercholesterolemia is usually treated by administration of a medicine, in combination with various types of dietary restrictions. However, the medicine predominantly utilized for hypercholesterolemia treatment, i.e., HMG-CoA reductase inhibitor for inhibiting the enzymatic action in the cholesterol synthesis pathway in the body, produces side effects such as rhabdomyolysis and liver functional impairment, and rebound phenomena. Also known is a medicine utilizing an ion exchange resin for adsorbing, in the intestines, bile acid having a high cholesterol content, and discharging the cholesterol from the body. However, the medicine needs to be taken in a great amount and has unpleasant taste. Moreover, long-term dietary restrictions are undesirable since the patient is forced to endure unpleasant meals.

It would therefore be ideal if the serum cholesterol level can be lowered while maintaining an ordinary diet, without dietary restrictions or administration of medicines having side effects such as rhabdomyolysis and liver functional impairement.

In view of the above status, various studies have been made on functional foods effective for lowering human serum cholesterol and improving lipid metabolism.

### BRIEF DESCRIPTION OF DRAWINGS

FIG. 1 is a graph showing the concentration dependency of the Bifidobacterium proliferation promoting effect.

FIG. 2 is a graph showing the Bifidobacterium proliferation promoting effect by combined use of specific vegetables.

FIG. 3 is a graph showing the change in mouse serum IgE level with time.

### DISCLOSURE OF THE INVENTION

An object of the present invention is to provide a Bifidobacterium proliferation promoting agent containing a Bifidobacterium proliferation promoting material which is free from the above drawbacks, has a high Bifidobacterium proliferation activity, is capable of being produced with ease at low cost, and is highly safe from the viewpoint of food hygiene; and a food and food material having a Bifidobacterium proliferation promoting action. Another object of the present invention is to provide a method for proliferating a Bifidobacterium.

The present inventors carried out extensive research to develop, from various natural products, materials having a high Bifidobacterium proliferation promoting activity. As a result, they found that vegetables and fruit such as broccoli, cabbage, spinach, komatsuna (Brassica rapa L.), takana (Brassica juncea Czern. et Coss. var. integrifolia Sinsk.), karashina (Brassica juncea Czern. et Coss.), cauliflower, kale, celery, parsley, lettuce and apple, have an extremely high Bifidobacterium proliferating activity, and accomplished the present invention. Further, the present inventors found that foods and food materials containing the above plants have an activity to promote Bifidobacterium proliferation, to thereby achieve the present invention.

Another object of the present invention is to provide a preventive or therapeutic agent for an allergy, and a food for preventing an allergy.

The present inventors found, as a result of extensive research, that vegetables and fruit such as broccoli, cabbage, spinach, komatsuna (Brassica rapa L.), takana (Brassica juncea Czern. et Coss. var. integrifolia Sinsk.), karashina (Brassica juncea Czern. et Coss.), cauliflower, kale, celery, parsley, lettuce and apple, have an inhibitory action against IgE increase which causes various allergies, in particular atopic dermatitis.

A further object of the present invention is to provide a food having a human serum cholesterol level lowering action, and a preventive or therapeutic agent for human hypercholesterolemia or arteriosclerosis without side effects such as liver functional impairment, the food and agent being free from the above drawbacks.

The present inventors had tested a mixed vegetable juice sold by their company to evaluate its cholesterol level lowering action, by administration in rats. However, no such action had been demonstrated. Therefore, it had been understood that the vegetable juice had no serum cholesterol level lowering action.

However, in a routine medical checkup and subsequent detailed checkup given in the inventors' company, a large number of stuff members were found to, have a remarkably lowered serum cholesterol level. Research on these stuff members revealed that they had not regularly drunk the vegetable juice before the routine checkup but started to drink, after the routine checkup, about one can per day of the juice sold in the company. Consequently, the vegetable juice was surprisingly found to have a human serum cholesterol level lowering action.

The present inventors carried out intense research concerning the effect of the mixed vegetable juice on human serum cholesterol, in order to find out which ingredient showed the action. Further, they extensively tested other edible plants for the action and found that broccoli, shepherd's purse and other edible plants, in particular, functional components in water-soluble fractions of the edible plants, have an inhibitory activity against human hepatocyte cholesterol synthesis, and a good serum cholesterol level lowering action and a good low-density lipoprotein cholesterol (LDL-Chol) level lowering action, on humans. Moreover, it was surprisingly found that combined use of cabbage, brussels sprouts, Japanese radish or Japanese radish leaves with the above edible plants having the human hepatocyte cholesterol synthesis inhibitory activity specifically enhance the human hepatocyte cholesterol synthesis inhibitory activity to thereby achieve a higher human serum cholesterol level lowering action and a higher human low-density lipoprotein cholesterol (LDL-Chol) level lowering action. Further, the present inventors found that a medicine containing the above edible plants is usable as a preventive or therapeutic agent for human hypercholesterolemia, or a preventive or therapeutic agent for human arteriosclerosis, both free from side effects such as liver functional impairment. The present invention has been accomplished based on the above findings.

The present invention provide the following foods, medicines and methods:

[1] A food containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

[1-A] A food according to Item [1], which contains at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

[1-B] A food according to Item [1], which contains at least one member selected from the group consisting of broccoli, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), Chinese cabbage, hinona (Brassica campestris var. akana), sugukina (Brassica campestris var. neosuguki), turnip, rape, cabbage, brussels sprouts, Japanese radish and Japanese radish leaves.

[1-A-i] A food according to Item [1-A], which has a Bifidobacterium proliferation promoting action.

[1-A-ii] A food according to Item [1-A], which has an allergy preventive action.

[1-B-i] A food according to Item [1-B], which has a human serum cholesterol lowering action.

[2] A medicine containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

[2-A] A medicine according to Item [2], which contains at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

[2-B] A medicine according to Item [2], which contains at least one member selected from the group consisting of broccoli, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), Chinese cabbage, hinona (Brassica campestris var. akana), sugukina (Brassica campestris var. neosuguki), turnip, rape, cabbage, brussels sprouts, Japanese radish and Japanese radish leaves.

[2-A-i] A medicine according to Item [2-A], which is a preventive or therapeutic agent for an allergy.

[2-B-i] A medicine according to Item [2-B], which is a preventive or therapeutic agent for human hypercholesterolemia.

[2-B-ii] A medicine according to Item [2-B], which is a preventive or therapeutic agent for human arteriosclerosis.

[3] A cosmetic having an action of alleviating rough skin conditions, containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

[3-A] A cosmetic having an action of alleviating rough skin conditions according to Item [3], which contains at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

[4] A method for preventing or treating a disease, comprising ingestion or administration of an effective amount of at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

[4-A] A method for preventing or treating a disease according to Item [4], which comprises ingestion or administration of an effective amount of at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

[4-B] A method for preventing or treating a disease according to Item [4], which comprises ingestion or administration of an effective amount of at least one member selected from the group consisting of broccoli, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), Chinese cabbage, hinona (Brassica campestris var. akana), sugukina (Brassica campestris var. neosuguki), turnip, rape, cabbage, brussels sprouts, Japanese radish and Japanese radish leaves.

[4-A-i] A method for preventing or treating a disease according to Item [4-A], wherein the disease is an allergy.

[4-B-i] A method for preventing or treating a disease according to Item [4-B], wherein the disease is human hypercholesterolemia.

[4-B-ii] A method for preventing or treating a disease according to Item [4-B], wherein the disease is human arteriosclerosis.

[5] A method for proliferating a Bifidobacterium, comprising contacting with the Bifidobacterium an effective amount of at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

[5-A] A method according to Item [5], which comprises contacting with the Bifidobacterium an effective amount of at least one member'selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

[6] A method for alleviating rough skin conditions, comprising applying to the skin an effective amount of a cosmetic containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

[6-A] A method according to Item [6], which comprises applying to the skin an effective amount of a cosmetic containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosugaki), turnip, rape, cabbage, spinach, komatsuna (Brassica rapa L.), celery, parsley, lettuce and apple.

The foods, medicines and cosmetics of the present invention will be described below in further detail.

First Invention [Food]

The food of the present invention contains at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves. These vegetables or fruit can be used either singly or in combination.

The above vegetables and fruit have a Bifidobacterium proliferation promoting action, an allergy preventive action and a human serum cholesterol lowering action. Therefore, the food of the invention, which contains at least one member selected from the above vegetables and fruit, has a Bifidobacterium proliferation promoting action, an allergy preventive action and a human serum cholesterol lowering action.

[1-A]

For use as a food having a Bifidobacterium proliferation promoting action or a food having an allergy preventive action, the food of the invention preferably contains at least member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce and apple.

Among these vegetables and fruit, more preferable are at least one member selected from the group consisting of broccoli, cabbage, spinach, komatsuna, takana, karashina, kale, cauliflower, celery, parsley, lettuce and apple; and particularly preferable are at least one member selected from broccoli, cabbage, takana, karashina, cauliflower and kale.

Moreover, it is preferable that the food [1-A] contains a combination of at least two member selected from broccoli, cabbage, spinach, komatsuna, celery, parsley, lettuce and apple. Preferred combinations are: broccoli and cabbage; apple and one of broccoli, kale and cabbage; spinach and one of broccoli, kale and cabbage; broccoli and kale; cabbage and kale.

These vegetables and fruit not only show a proliferation promoting action on almost all strains of Bifidobacteria, but also promote proliferation of Lactobacilli, which are known to have advantageous actions like Bifidobacteria. On the other hand, these vegetables and fruit scarcely show a proliferation promoting action on Clostridium perfringens, a toxic bacterium. That is, these vegetables and fruit have a selective proliferation promoting action on bacteria.

According to the invention, edible parts of these vegetables or fruit are usually utilized, unless otherwise specified. For example, spinach root, which is usually inedible, is not used in the invention. In the case of apple, apple fruit is utilized.

In the present invention, the vegetables or fruit may be used in a raw state, or as thermally sterilized. Alternatively, the raw or thermally sterilized vegetables or fruit may be dried with hot air or freeze-dried, to prepare a film, plate, block or like solid product. Also, a powder prepared by grinding the solid product is usable.

Further alternatively, the raw or thermally sterilized vegetables or fruit may be crushed or squeezed in order to use the resultant product as a whole, or to use only the vegetable or fruit juice obtained. Also usable are a concentrate of the vegetable or fruit juice, or a powder, granules, tablets or like product prepared by drying and processing the concentrate. Further usable are a juice obtained from the raw or thermally sterilized vegetables or fruit using a suitable filter cloth or the like, and a supernatant fluid obtained by centrifugation of the filtrate. The supernatant fluid may be dried with hot air or freeze-dried to obtain a film, plate block or like solid product. The solid product may be ground to obtain a powder.

Thus, for use in the invention, the above vegetables or fruit may be processed by a conventional process comprising any combination of operations such as washing, sorting, peeling, coring, crushing, squeezing, filtration, separation, concentration, heating, cooling, homogenization and drying.

The food [1-A] may consist solely of at least one member selected from the above vegetables and fruit, or may contain other ingredients commonly used for foods.

The amount of the other ingredients to be added is not limited as long as the Bifidobacterium proliferation promoting action or allergy preventive action is exhibited, but is, for example, about 0.001 to 99.99 wt. % based on the total amount after addition of the other ingredients.

Examples of usable other ingredients include vegetables or fruits other than those listed above, such as lemon, orange, carrot, Japanese radish leaves, pumpkin, sweet pepper, and sweet potato. They are used in the form of a juice, a concentrate, granules or the like obtained by the same process as for the above vegetables or fruit.

Further, additives commonly used for preparation of foods or food materials may be used as other ingredients, as long as they do not hinder the Bifidobacterium proliferation promoting action or allergy preventive action. Specific examples of usable additives include apple fiber, corn fiber, alginic acid and like fibers; lactose, starch and like excipients; sucrose, maltose, sorbitol, mannitol, oligosaccharide and like sweeteners; vitamins, calcium and like nutritional supplements; thickeners; condiments; colors; etc.

When the food of the invention is used as a food having a Bifidobacterium proliferation promoting action, a Bifidobacterium may be added to the food in advance. Addition of a Bifidobacterium is advantageous because the intestinal flora improves fast.

The other vegetables or fruit and additives can be selected according to the intended use and form of the food, the user's taste and other factors, and used in a suitable combination.

Where necessary, the food [1-A] can be made into or mixed in a desired food product such as: a powder; granules; tablets; capsules; candies; chewing gum; nougat; chocolate; jelly; biscuits; cake; bread; noodles; a beverage; vegetable juice; spread; lactic fermenting beverage, miso(soybean paste), yogurt or like fermented food; fish cake, boiled fish paste or like sea food paste; ham, sausage, processed milk, cheese or like domestic animal food product; or gravy, dressing, sauce, soy sauce or like seasoning. Among these food products, a beverage, in particular vegetable juice, is preferred as the form of the food of the invention.

The food [1-A] can be used as a food material for preparing a desired food product such as the foods mentioned above. The food material may be used singly to prepare a food product, or may be combined with other food materials to prepare a food product.

The food [1-A] may be ingested as a mixture with a prepared food such as dehydrated soup or powdered juice, or as a solution or dispersion in canned juice or the like.

When the food of the invention is used as a food having a Bifidobacterium proliferation promoting action, i.e., the food [1-A-i], the intake of the food can be determined according to the form of the food, the age, weight and sex of the human or animal to be given the food, purpose of intake, and other factors. The intake of the food is not limited as long as the Bifidobacterium proliferation promoting action is achieved. It is usually suitable that the adult daily intake of the food is about 5 to 1000 ml when the food is in a liquid form, or about 0.1 to 100 g when the food in a solid form. The daily intake is preferably divided into several servings. The intake of the food used as a food material may be the same as above.

When the food of the invention is used as an allergy preventive food, i.e., as the food [1-A-ii], the intake of the food is not limited and may be determined according to the form of the food, the age, weight and sex of the human or animal to be given the food, purpose of intake, and other factors. Usually, the adult daily intake of the food is determined so that the intake of the above vegetables or fruit becomes about 0.01 to 500 g daily, preferably about 0.1 to 150 g daily, more preferably about 7 to 30 g daily, as measured in a raw state. The daily intake may be ingested in a single serving or in 2 to 4 divided servings.

The food [1-A] may be taken at any time of day, for example, before, after or between meals. It is preferable that the food is taken on an empty stomach or before bedtime.

As discussed before, the above vegetables and fruit such as broccoli, cabbage and apple have a high Bifidobacterium proliferation promoting action and are capable of selectively proliferating Bifidobacteria, beneficial intestinal bacteria. Accordingly, the food of the invention containing the vegetables or fruit also has a high Bifidobacterium proliferation promoting effect and is capable of selectively proliferating Bifidobacteria, beneficial intestinal bacteria.

Further, the food of the invention shows, as a result of the Bifidobacteria proliferation in the intestines, effects such as improvement of the intestinal flora, promotion of beneficial intestinal bacterium proliferation, prevention of harmful intestinal bacterium proliferation, improvement of the intestinal environment, improvement of fecal property, prevention of intestinal putrefaction, increase of fecal weight, improvement in defecation frequency, maintenance of good gastro intestinal conditions, and controlling intestinal functions. Further, the improvement of the intestinal flora by intestinal Bifidobacterium proliferation produces anticancer effect, anti-aging effect, immunostimulating effect and like effects. The improvement of the intestinal flora will also lead to prevention and alleviation of rough skin conditions.

The food of the invention has an effect of inhibiting IgE increase, and therefore is expected to have a preventive effect against a variety of allergies such as atopic dermatitis, urticaria, bronchial asthma, allergic rhinitis, pollinosis and allergic conjunctivitis. Particularly expected of the food of the invention are effects of alleviating symptoms of allergic dermatitis accompanied by IgE increase, for example a preventive or therapeutic effect on atopic dermatitis.

Therefore, the food of the invention can be used as a health food, a functional food, a food for specified health use, a food for sick people, etc.

[1-B]

When the food of the present invention is used as a food having a human serum cholesterol lowering action, the food preferably contains at least one member selected from the group consisting of broccoli, shepherd's purse, suzushiro, hatazao, tagarashi, woad, oobatanetsukebana, yamagarashi, watercress, takana, karashina, Japanese horseradish, yuriwasabi, Chinese cabbage, hinona, sugukina, turnip, rape, cabbage, brussels sprouts, Japanese radish and Japanese radish leaves.

These edible plants have a hepatocyte cholesterol synthesis inhibitory activity, which is an action mechanism to reduce the human serum cholesterol level most effectively, and to reduce the level of low-density lipoprotein cholesterol (LDL-Chol), a harmful cholesterol. The food of the invention, which contains these plants, have a high human serum cholesterol level lowering action and a high human LDL-Chol level lowering action. Namely, the food of the invention is effective for improving lipid metabolism. The food [1-B] can be ingested as a health food, a functional food, a food for specified health use, a food for sick people, etc.

The food [1-B] is limited to human use, and is not intended to use as a food or feed for non-human animals (dogs, cats, rabbits, mice, rats, etc.).

It is particularly preferable that the food [1-B] contains at least one member selected from the group consisting of broccoli, shepherd's purse, suzushiro, hatazao, tagarashi, woad, oobatanetsukebana, yamagarashi, watercress, takana, karashina, Japanese horseradish, yuriwasabi, Chinese cabbage, hinona, sugukina, turnip and rape.

Two or more of the above listed vegetables can be used in combination. Preferred combinations are: at least one of cabbage, brussels sprouts, Japanese radish and Japanese radish leaves, and at least one of broccoli, shepherd's purse, suzushiro, hatazao, tagarashi, woad, oobatanetsukebana, yamagarashi, watercress, takana, karashina, Japanese horseradish, yuriwasabi, Chinese cabbage, hinona, sugukina, turnip and rape.

When only one of the vegetables is used, broccoli is most preferable. Specific examples of preferred combinations are: cabbage and broccoli; cabbage, Japanese radish leaves and broccoli; and cabbage, broccoli and Chinese cabbage.

For use in the food [1-B], the above plants can be processed in the same manner as for the food [1-A].

The total content of these plants (vegetables) in the food varies according to the kind of food to be prepared and differences among the users (individual differences, racial differences, etc.), and is not limited as long as the human serum cholesterol lowering effect can be achieved. It is suitable that the total content is about 0.01 to 100 wt. %, preferably about 1.0 to 99.9 wt. %.

A total content less than 0.01 wt. % is liable to result in insufficient lipid metabolism improving effect, hence undesirable.

The food [1-B] may contain other ingredients in addition to the above plants, as long as the human serum cholesterol lowering effect is not hindered.

Usable other ingredients include kale, soybeans, oats, barley and like plants known to have a cholesterol lowering action. Also, other plants commonly used in foods, such as celery, lettuce, spinach, komatsuna (Brassica rapa L.), parsley, apple and lemon, which have been processed in the above manner, can be added.

In particular, the food [1-B] preferably contains at least one member selected from celery, lettuce, spinach, komatsuna, parsley, apple and lemon, in addition to the combination of broccoli and one of cabbage and Japanese radish leaves.

The food [1-B] may contain, as additional ingredients, additives commonly used in the food industry, such as starches, lactose, cellulose, dextrin, sugar alcohols, mucopolysaccharides and like excipients; sucrose, maltose, sorbitol, mannitol, oligosaccharides and like sweeteners; and vitamins, calcium and like nutritional supplements.

The food [1-B] can be prepared by adding, to the starting materials for preparing a food, the plants processed in the above manner, or the processed plants diluted with a excipient or part of the starting materials, or a solution or dispersion of the processed plants in ethanol, water or the like, at an appropriate stage of the production. The resulting mixture is then mixed in a conventional manner, followed by an ordinary process for preparing the food. The mixing step may be carried out with heating.

Alternatively, the food [1-B] may be prepared by adding the plants processed in the above manner or a mixture or solution of the processed plants, to a prepared food product, followed by mixing in a conventional manner.

The food [1-B] is limited to human use, and is not intended to be used as a food or feed for non-human animals (dogs, cats, rabbits, rats, mice, etc.) The food [1-B] is not limited in form, and may be made into any of the forms described for the food [1-A] by a conventional process. Like the food [1-A], the food [1-B] can be used as a food material. Alternatively, the food may be ingested as a mixture with a prepared food product, such as dehydrated soup or powdered juice, or as a solution or dispersion in canned juice.

The intake of the food [1-B] can be determined according to the form of the food, the age, weight, sex and health conditions of the user, and purpose of intake, without limitation. It is usually preferable that the adult daily intake of the food is about 5 to 1,000 ml when the food is in a liquid form, or about 0.1 to 100 g when the food is in a solid form. The daily intake is preferably divided into several servings.

The food may be taken at any time of day, i.e., before, after or between meals. Intake on before breakfast, lunch or evening meal, or intake on an empty stomach is particularly effective.

Ingestion of the food [1-B] lowers the human serum cholesterol level without dietary restrictions or programmed exercises.

The food [1-B] can be used in combination with a conventional preventive or therapeutic agent for hypercholesterolemia, such as Mevalotin. In the combined use, the amount of the conventional agent can be reduced, since the cholesterol lowering action of the food of the invention will compensate the reduction. Thus, the combined use with the food of the invention is advantageous in a long term prevention or treatment, from the viewpoint of side effects.

Second Invention [Medicine]

The medicine of the present invention contains at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce, apple, Chinese cabbage, brussels sprouts, Japanese radish and Japanese radish leaves. In the medicine of the invention, these vegetables or fruit may be used singly or in combination.

The medicine of the invention which contains at least one member selected from the above vegetables and fruit, can be used as a preventive or therapeutic agent for an allergy, a preventive or therapeutic agent for human hypercholesterolemia, or a preventive or therapeutic agent for human arteriosclerosis.

[2-A]

The medicine of the invention has an inhibitory effect against IgE increase, as mentioned above for the food [1-A]. Thus, the medicine is expected to have a preventive or therapeutic effect on a wide variety of allergies such as atopic dermatitis, urticaria, bronchial asthma, allergic rhinitis, pollinosis and allergic conjunctivitis. Particularly expected of the medicine of the invention are alleviating effects on symptoms of allergic dermatitis accompanied by IgE increase, for example, a preventive or therapeutic effect on atopic dermatitis.

When the medicine of the invention is used as a preventive or therapeutic agent for an allergy, the medicine preferably contains at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce and apple as same as the food [1-A]. Among these vegetables and fruit, more preferred kinds and preferred combinations are the same as for the food [1-A].

The vegetables or fruit for use in the medicine [2-A] can be processed in the same manner for the food [1-A].

The medicine [2-A] may consist solely of at least one member selected from the above vegetables and fruit, or may contain other ingredients.

The amount of other ingredients to be added is not limited as long as the preventive or therapeutic action for an allergy is exhibited. The amount is, for example, about 0.001 to 99.99 wt. % based on the total amount after addition of the other ingredients.

Examples of other ingredients include vegetables or fruit other than those listed above, such as lemon, orange, carrot, Japanese radish leaves, pumpkin, sweet pepper and sweet potato. These vegetables or fruit can be processed in the same manner as for the above vegetables or fruit, and added in the form of juice, concentrate, granules or the like.

Also usable as the other ingredients are additives conventionally used for production of medical preparations, such as excipients, expanders, binders, wetting agent, disintegrators, surfactants, lubricants, dispersants, buffers, preservatives, solubilizers, anticeptics, flavors and stabilizers.

The other vegetables or fruit and additives can be suitably selected and combined, according to the intended use and form of the medicine [2-A], taste of the user, and other factors.

When the medicine of the invention is used in the form of a medical preparation, a variety of dosage forms can be employed according to the intended therapy. Typical examples of dosage forms include tablets, pills, granules, capsules, troches and other solid preparations; powders for internal application, powders for external application and like powder preparations; solutions, suspensions, emulsions, syrups, lotions, aerosols, infusions, decoctions and other liquid preparations; ointments and other cream preparations; and cataplasms.

The medical preparation can be produced by a conventional process for producing the intended preparation.

The method of administration of the preparation is not limited. Thus, the preparation may be administered, for example, orally or transdermally, according to the form of the preparation, the age, sex and other characteristics of the patient, the severity of the disease, and other factors.

Further, the method of administration may one comprising applying a liquid preparation such as an infusion or decoction to a mask by immersion, coating, spraying or like means; drying the mask; and applying the mask to the patient (in particular a patient with pollinosis).

The daily dose of the preparation is selected according to the form of the preparation, the age, sex and other characteristics of the patient, the severity of the disease and other factors, etc. Usually, it is suitable that the preparation is administered in such an amount that the adult daily dose of the above vegetables or fruit becomes about 0.01 to 500 g, preferably about 0.1 to 150 g, more preferably about 7 to 30 g, as measured in a raw state. The daily dose may be administered in a single dose or in several divided doses.

[2-B]

The above edible plants such as broccoli and shepherd's purse have an inhibitory activity against human hepatocyte cholesterol synthesis, and thus possesses a human serum cholesterol lowering effect. Therefore, a medicine containing at least one of these plants is usable as a preventive or therapeutic agent for human hypercholesterolemia, or as a preventive or therapeutic agent for human arteriosclerosis.

The medicine [2-B] is limited to human use, and is not intended to be used as a medicine for non-human animals (dogs, cats, rabbits, rats, mice, etc.), particularly as a preventive or therapeutic agent for hypercholesterolemia or arteriosclerosis of non-human animals.

When the medicine of the invention is used as a preventive or therapeutic agent for human hypercholesterolemia or as a preventive or therapeutic agent for human arteriosclerosis, the medicine preferably contains at least one member selected from the group consisting of broccoli, shepherd's purse, suzushiro, hatazao, tagarashi, woad, oobatanetsukebana, yamagarashi, watercress, takana, karashina, Japanese horseradish, yuriwasabi, Chinese cabbage, hinona, sugukina, turnip, rape, cabbage, brussels sprouts, Japanese radish and Japanese radish leaves. Among these edible plants, more preferred kinds and preferred combinations for the medicine [2-B] are the same as for the food [1-B].

The vegetables used can be processed in the same manner as for the food [1-A].

The medicine [2-B] may contain a suitable amount of additives conventionally used for production of medical preparations, such as excipients, expanders, binders, wetting agents, disintegrators, surfactants, lublicants, dispersants, buffers, preservatives, solubilizers, anticeptics, flavors and stabilizers.

The amount of the additives is not limited as long as the contemplated effects are not hindered, and may be, for example, 0.001 to 90.0 wt. %, preferably about 0.01 to 70.0 wt. %.

The medicine [2-B] may be used in a desired form such as a solution, tablets, granules or pills, according to the purpose. The medicine can be prepared by processing plants such as broccoli in the same manner as for the food [1-A], and making the processed plants into a preparation by a conventional process.

The method of administration of the medicine [2-B] is not limited. Thus, the medicine may be administered, for example, orally or transdermally, according to the form of the preparation, the age, sex and other characteristics of the patient, the severity of the disease, and other factors.

The dose of the medicine [2-B] (preventive or therapeutic agent) can be suitably selected without limitation, according to the form of the medicine, the age, weight and sex of the human patient, the purpose and other factors. It is usually suitable that the adult daily dose of the medicine is about 5 to 1,000 ml when the medicine is a liquid preparation, or about 0.1 to 100 g when the medicine is a solid preparation. The daily dose is preferably administered in several divided doses. The medicine may be administered at any time of day. Administration on before breakfast, lunch or evening meal, or on an empty stomach is particularly preferable.

The preventive or therapeutic agent for human hypercholesterolemia and the preventive or therapeutic agent for human arteriosclerosis of the invention do not produce side effects such as rhabdomyolysis and liver functional impairment.

Third Invention [Cosmetic]

The third invention provides a cosmetic having an effect of alleviating rough skin conditions, containing at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce, apple, Chinese cabbage, brussels sprouts, Japanese radish and Japanese radish leaves.

The cosmetic of the invention has an effect of alleviating rough skin conditions caused by various factors (such as atopic dermatitis, urticaria, contact dermatitis and like allergies).

The cosmetic of the invention may consist solely of at least one of the above vegetables and fruit, or may contain other vegetables or the like.

As the vegetables or fruit to be contained in the cosmetic of the invention, preferred is at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce and apple. For use in the cosmetic of the invention, more preferred kinds, preferred combinations, amount and processing method of the vegetables or fruit, and kinds, combinations and amount of other usable vegetables, fruit and the like are the same as for the food [1-A] or the medicine [2-A].

The form of the cosmetic can be selected from various forms according to the purpose. Specific examples include soaps, facial cleaning preparations, skin lotions, milky lotions, foundations, lipsticks, lip creams, cleansing creams, massage creams, packs, hand creams, hand powders, body shampoos, body lotions, body creams and bath cosmetics.

The cosmetic of the invention can be prepared by a conventional process using the above vegetables or fruit together with a carrier suitable for the form of the cosmetic to be prepared.

The carrier for use in the cosmetic of the invention can be selected from conventional carriers, according to the form of the cosmetic. Usable carriers include binders, surfactants, humectants, fillers, expanders, moistening agents and like diluents and excipients.

The cosmetic of the invention may further contain, where necessary, anticeptics, coloring matters, preservatives, antioxidants, aromatics and the like.

The cosmetic of the invention can be used by a conventional method suitable for the form of the cosmetic.

The amount of the cosmetic to be used is determined according to the form of the cosmetic, the degree of skin roughening and other factors, without limitation. The cosmetic can be used, for example, in such an amount that the daily amount of the above vegetables or fruit to be used becomes about 0.01 to 10 g, when measured as raw vegetables or fruit. The daily amount may be used in a single portion or in 2 to 4 divided portions.

Fourth Invention [Method for Preventing or Treating a Disease]

The present invention also provides a method for treating or preventing a disease, comprising ingestion or administration of an effective amount of at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce, apple, Chinese cabbage, brussels sprouts, Japanese radish and Japanese radish leaves.

In the method of the invention, the vegetables or fruit are ingested or administered in the form of, for example, a food or a medicine.

[4-A]

If the method of the invention is a method for preventing or treating an allergy, it is preferable to ingest or administer an effective amount of at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce and apple. As to the vegetables or fruit to be ingested or administered in the method [4-A], more preferred kinds, preferred combinations, processing method, amount in a composition to be administered, etc. are the same as for the food [1-A] or the medicine [2-A].

In the method [4-A], the above vegetables or fruit may be ingested or administered together with other vegetables, additives or the like. Usable kinds, combinations, and amount to be added of the other vegetables, additives or the like may be the same as for the food [1-A] or the medicine [2-A]. The form, intake or dose, frequency of ingestion or administration, and method of ingestion or administration, of the food or preparation may be the same as for the food [1-A] or the medicine [2-A].

[4-B]

If the method of the invention is a method for preventing or treating human hypercholesterolemia or human arteriosclerosis, it is preferable to ingest or administrate an effective amount of at least one member selected from the group consisting of broccoli, shepherd's purse, suzushiro, hatazao, tagarashi, woad, oobatanetsukebana, yamagarashi, watercress, takana, karashina, Japanese horseradish, yuriwasabi, Chinese cabbage, hinona, sugukina, turnip, rape, cabbage, brussels sprouts, Japanese radish and Japanese radish leaves. As to the vegetables or fruit to be ingested or administered in the method [4-B], more preferred kinds, preferred combinations, processing method, amount in a composition to be administered, etc. are the same as for the food [1-B] or the medicine [2-B].

In the method [4-B], the above vegetables or fruit may be ingested or administered together with other vegetables, additives or the like. Usable kinds, combinations and amount of the other vegetables, additives or the like may be the same as for the food [1-B] or the medicine [2-B]. The form, method of ingestion or administration, intake or dose, and frequency of ingestion or administration of the food or preparation may be the same as for the food [1-B] or the medicine [2-B].

Fifth Invention [Method for Proliferating Bifidobacterium]

The present invention also provides a method for proliferating a Bifidobacterium, comprising contacting with the Bifidobacterium an effective amount of at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce, apple, Chinese cabbage, brussels sprouts, Japanese radish and Japanese radish leaves.

The method of the fifth invention is a method for proliferating a Bifidobacterium utilizing the Bifidobacterium proliferating activity of the above vegetables or fruit (hereinafter the vegetables or fruit being sometimes referred to as “Bifidobacterium proliferation promoting material”).

In the method of the fifth invention, it is particularly preferable to use at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce and apple.

More preferred kinds, preferred combinations, and processing method of the vegetables or fruit (Bifidobacterium proliferation promoting material) such as broccoli, may be the same as for the food [1-A].

In the method of the fifth invention, vegetables other than the Bifidobacterium proliferation promoting materials, additives or the like can be used. Usable kinds, combinations and amount of the other vegetables, additives or the like, may be the same as for the food [1-A] or the medicine [2-A].

In one embodiment of the invention, the Bifidobacterium proliferation promoting material is contacted with a Bifidobacterium, for example, by adding the Bifidobacterium proliferation promoting material to a system containing the Bifidobacterium, or by adding the Bifidobacterium to a system containing the Bifidobacterium proliferation promoting material. Another embodiment comprises adding the Bifidobacterium proliferation material and Bifidobacterium to a system not containing a Bifidobacterium.

In the method of the invention, two or more kinds of the Bifidobacterium proliferation promoting material may be used in combination. For example, 1 to 24 hours after addition of one kind of Bifidobacterium proliferation promoting material, another kind of Bifidobacterium proliferation promoting material may be further added.

The method of the invention can be carried out as follows, in the case where a Bifidobacterium is proliferated in a culture medium.

The amount of the Bifidobacterium proliferation promoting material for use in the invention can be selected according to the purpose of use, kind of the Bifidobacterium proliferation promoting material and other factors, and is not limited as long as the Bifidobacterium proliferation promoting effect is exhibited. For example, the material may be used, as a vegetable juice or fruit juice, in an amount of about 0.01 to 100 wt. %, preferably about 1 to 100 wt. %, based on the total weight of the culture medium after addition of the material. The Bifidobacterium proliferation promoting material by itself can be used as a culture medium. In that case, the material accounts for 100 wt. % of the culture medium.

As other ingredients, agars, amino acids, sodium chloride, yeast extracts and like culture media may be previously added to the Bifidobacteria proliferation promoting material. The amount of these culture media to be added is not limited as long as the contemplated effect is not hindered, and is, for example, about 0.001 to 99.99 wt. % based on the total amount after addition of the culture media as other ingredients. The Bifidobacterium proliferation promoting material to which the culture media have been previously added, can be used as a culture medium or as an additive for another culture medium.

For Bifidobacterium proliferation, incubation is carried out at about 15 to 40° C., in particular about 30 to 37° C., for about 5 to 200 hours, in particular about 24 to 72 hours, after addition of the Bifidobacterium proliferation promoting material.

Further, the Bifidobacterium proliferation promoting material such as broccoli or cauliflower may be ingested as a food or administered as a medicine to contact the material with intestinal Bifidobacteria of humans or non-human animals such as dogs, cats and rats to proliferate the intestinal Bifidobacteria, in a similar as in the proliferation in a culture medium.

For proliferating intestinal Bifidobacteria by ingestion of the Bifidobacterium proliferation promoting material as a food or by administration of the material as a medicine, the intake or dose of the material can be suitably determined according to the form of the food or medicine, the age, weight, sex and other characteristics of the human or non-human animals to be given the material. Thus, the intake or dose is not limited as long as the Bifidobacterium proliferation promoting action can be exhibited. It is preferable that the material is ingested or administered as a food in an adult daily intake of about 5 to 1000 ml when in a liquid form, or about 0.1 to 100 g when in a solid form, to proliferate a Bifidobacterium. The daily intake may be ingested or administered in a single serving or dose or in several divided servings or doses.

Alternatively, the material may be ingested or administered in an adult daily intake of about 0.1 to 500 g, either in a single serving or dose in several divided servings or doses.

The method of the invention which promotes proliferation of Bifidobacteria in the intestines, is expected to produce the same effect as of the ingestion of the food [1-A], such as improvement of the intestinal flora. Further, the method of the invention is expected to achieve anticancer effect, anti-aging effect, immunostimulant effect, effect of preventing and alleviating rough skin conditions, etc., as a result of the improvement of intestinal flora by proliferation of intestinal Bifidobacteria.

Sixth Invention [Method for Alleviating Rough Skin Conditions]

The present invention further provides a method for alleviating rough skin conditions, comprising applying to the skin an effective amount of a cosmetic containing at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce, apple, Chinese cabbage, brussels sprouts, Japanese radish and Japanese radish leaves.

The method of the sixth invention alleviates rough skin conditions caused by atopic dermatitis, urticaria, contact dermatitis and various other allergies.

As the vegetables or fruit contained in the cosmetic for use in the method of the invention, preferred is at least one member selected from the group consisting of broccoli, cauliflower, kale, shepherd's purse, suzushiro, hatazao, tagarashi, woad, oobatanetsukebana, yamagarashi, watercress, takana, karashina, Japanese horseradish, yuriwasabi, hinona, sugukina, turnip, rape, cabbage, spinach, komatsuna, celery, parsley, lettuce and apple.

As to the vegetables or fruit in the cosmetic for use in the method of the sixth invention, more preferred kinds, preferred combinations, processing method, and amount in a composition to be applied may be the same as for the cosmetic of the third invention. Also, the form of the cosmetic may be the same as in the third invention.

The effective amount (amount to be applied) of the cosmetic for use in the method of the invention may be the same as for the cosmetic of the third invention.

In the method of the sixth invention, the cosmetic is contacted with the skin in an ordinary manner suitable for the form of the cosmetic, such as spreading, patting or packing. The cosmetic of the invention is used preferably after washing the face, after a bath, or before bedtime.

The vegetables or fruit to be used as active ingredients of the food or medicine of the present invention, have been used as food throughout ages, and therefore can be ingested or administered safely over a long term, for prophylactic or therapeutic purposes.

### BEST MODE FOR CARRYING OUT THE INVENTION

The following Test Examples and Comparative Test Examples are provided to illustrate the present invention in further detail. In these examples, percentages are all by weight (w/w %) unless otherwise specified.

### Test Example 1

Vegetables and fruit such as broccoli, cabbage and apple were tested for proliferation promoting activity on a Bifidobacteriium.

(1) Preparation of Test Sample

The vegetables and fruit shown in Table 1 were crushed and centrifuged, and the supernatants were collected.

(2) Test Strain

A: Bifidobacterium longum JCM1217

(3) Test Method

(3-1) Preparation of Precultured Medium of Test Strain

A loopful of the test strain was taken from a colony developed on a GAM agar medium (Nissui Pharmaceutical Co.) and inoculated onto a GAM broth medium, followed by incubation at 37° C. for 24 hours under anaerobic conditions (H₂: 10%, CO₂: 5%, N₂: 85%). The incubated culture was then diluted with purified water to a turbidity of 0.5 (wavelength: 620 nm).

(3-2) Test of Proliferation Promoting Effect

The basal media used were GAM broth media (Nissui Pharmaceutical Co.) Bacteria were removed from the test sample solutions through a sterilization filter (0.45 μm) and the solutions were added individually to the basal media to a concentration of 25 wt. %, to obtain test culture media.

The test culture media were each inoculated with the precultured medium of the test strain obtained in (3-1) to a concentration of 1%. After measurement of the turbidity, incubation was carried out anaerobically at 37° C. for 24 hours, followed by measurement of the turbidity of the incubated culture media.

A control culture meddium was prepared in the same manner as the preparation of the test culture media, with the exception that purified water was used in place of the test sample solutions.

The test strain proliferation promotion ratio of each test sample was calculated from the measurements of turbidity, according to the following equation:

Proliferation promotion ratio (times)=(T₂₄−T₀)/(C₂₄−C₀)

T₂₄: Turbidity of test culture medium after 24-hour incubation

T₀: Turbidity of test culture medium before incubation

C₂₄: Turbidity of control culture medium after 24-hour incubation

C₀: Turbidity of control culture medium before incubation

(4) Test Result

Table 1 shows the test results.

TABLE 1

| | Proliferation |
|---|---|
| | promotion |
| Test sample | ratio (times) |
| Apple | 6.2 |
| Broccoli | 8.5 |
| Celery | 4.8 |
| Cabbage | 8.7 |
| Lettuce | 4.2 |
| Spinach | 5.1 |
| Parsley | 3.1 |
| Komatsuna | 7.0 |

On Bifidobacterium longum, the vegetables and fruit shown in Table 1 exhibited a proliferation effect at least three times higher than that of the control medium. In particular, broccoli, cabbage and komatsuna exhibited an extremely high effect.

### Test Example 2

The vegetables which showed a particularly high proliferation promoting effect in Test Example 1 were tested for proliferation promoting effect on Bifidobacteria other than Bifidobacterium longum.

(1) Preparation of Test Sample

Test samples were prepared in the same manner as in Test Example 1.

(2) Test Strain

A: Bifidobacterium longum JCM 1217

B: Bifidobacterium bifidum JCM 1255

C: Bifidobacterium infantis JCM 1222

D: Bifidobacterium breve JCM 1192

E: Bifidobacterium adolescentis JCM 1275

F: Bifidobacterium pseudolongum ATCC 25526

G: Bifidobacterium animalis ATCC 25527

(3) Test Method

The test was performed in the same manner as in Test Example 1.

(4) Test Result

Table 2 shows the test results.

TABLE 2

| | Proliferation promotion ratio (times) | | |
|---|---|---|---|
| Test | Test sample | | |
| strain | Cabbage | Broccoli | Komatsuna |
| A | 8.7 | 8.5 | 7.0 |
| B | 8.5 | 8.2 | 6.8 |
| C | 8.5 | 8.2 | 6.9 |
| D | 8.8 | 8.5 | 7.2 |
| E | 8.5 | 8.2 | 6.8 |
| F | 8.5 | 8.2 | 6.8 |
| G | 8.5 | 8.0 | 6.7 |

As shown in Table 2, cabbage, broccoli and komatsuna, in particular cabbage and broccoli, showed an extremely high proliferation promoting effect on all of the seven test Bifidobacterium strains.

The above results demonstrate that these vegetables have a remarkably high proliferation promoting effect on Bifidobacteria, regardless of the strains.

### Test Example 3

Test samples prepared from cabbage and broccoli were added individually to culture media to obtain graded concentrations from 1 to 75 wt. %, for testing the concentration dependency of the Bifidobacterium proliferating effect of these vegetables. Further, the effect of combined use of cabbage and broccoli was tested.

(1) Preparation of Test Sample

Test samples were prepared in the same manner as in Test Example 1.

(2) Test Strain

A: Bifidobacterium longum JCM 1217

(3) Test Method

The test was performed in the same manner as in Test Example 1.

(4) Test Result

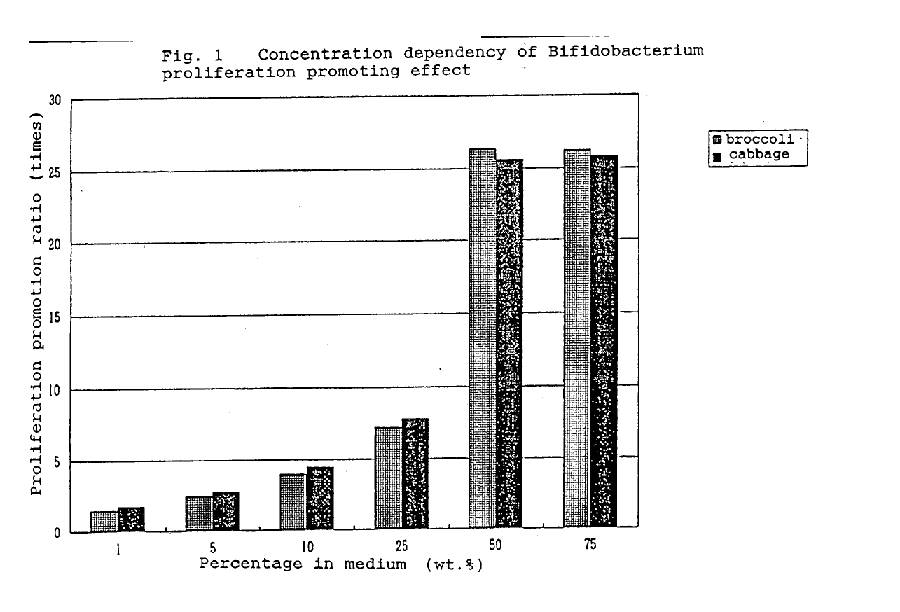

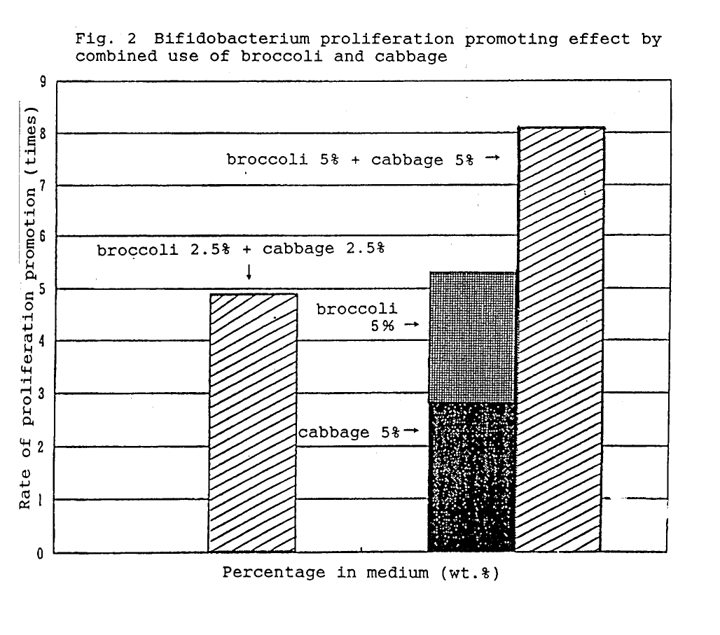

FIGS. 1 and 2 show the test results. FIG. 1 demonstrates that a sufficient Bifidobacterium proliferation promoting action can be achieved by addition of each test sample at a concentration of 0.01 to 100 wt. %, preferably 1 to 50 wt. %, based on the culture medium. It should be noted, however, higher concentrations do not hinder the effect of the invention. FIG. 2 reveals that the combined use of broccoli and cabbage exhibits a Bifidobacterium proliferation effect, and that combined use (for example, 5 wt. % of broccoli and 5 wt. % of cabbage) achieves a higher Bifidobacterium proliferation promoting effect than single use of each vegetable.

### Test Example 4

Broccoli and cabbage were tested for effect on proliferation of an intestinal bacterium other than Bifidobacteria.

(1) Preparation of Test Sample

Test samples were prepared in the same manner as in Test Example 1.

(2) Test Strains

A: Bifidobacterium longum JCM 1217

X: Clostridium perfringens JCM 1290

(3) Test Method

The test was performed in the same manner as in Test Example 1.

(4) Test Result

Table 3 shows the test results.

TABLE 3

Proliferation promotion ratio (time)

| | Test sample | |
|---|---|---|
| Test strain | Broccoli | Cabbage |
| A | 8.5 | 8.7 |
| X | 1.8 | 1.4 |

Table 3 shows that the above test samples exhibit a remarkable proliferation promoting effect on the Bifidobacterium, but show no significant proliferation promoting effect on Clostridium perfringens JCM 1290, a harmful bacterium.

### Test Example 5

The test samples shown in Table 4 were tested for Bifidobacterium proliferation promoting effect.

(1) Preparation of Test Sample

Test Samples 1 to 6 shown in Table 4 were prepared by processing the vegetables in the same manner as in Test Example 1. The numerical values presented in Table 4 indicate percentages by weight of the vegetables.

TABLE 4

| | Test sample | | | | | |
|---|---|---|---|---|---|---|
| | 1 | 2 | 3 | 4 | 5 | 6 |
| Broccoli | 100 | — | — | — | — | 75 |
| Cabbage | — | 100 | — | — | — | 25 |
| Takana | — | — | 100 | — | — | — |
| Karashina | — | — | — | 100 | — | — |
| Cauliflower | — | — | — | — | 100 | — |
| Total | 100 | 100 | 100 | 100 | 100 | 100 |

(2) Test Strains

A: Bifidobacterium longum JCM 1217

(3) Test Method

(3-1) Preparation of Precultured Medium of Test Strain

A precultured medium of the test strain was prepared in the same manner as in Test Example 1 (3-1).

(3-2) Test of Proliferation Effect

Bacteria were removed from the test samples shown in Table 4 through a sterilization filter (0.45 μm) and each of the test samples were inoculated with the precultured medium to a concentration of 1%. After measurement of turbidity, the inoculated media were incubated anaerobically at 37° C. for 24 hours. Then, the turbidity of the incubated media was measured under the same conditions as in Test Example 1.

Bifidobacterium proliferation was calculated according to the following equation:

Bifidobacterium proliferation=T₂₄−T₀

T₂₄: Turbidity of test medium after 24-hour incubation

T₀: Turbidity of test medium before incubation

(4) Test Result

Table 5 shows the test results.

TABLE 5

| | Bifidobacteria |
|---|---|
| | proliferation |
| Test Sample 1 | 0.526 |
| Test Sample 2 | 0.537 |
| Test Sample 3 | 0.400 |
| Test Sample 4 | 0.350 |
| Test Sample 5 | 0.380 |
| Test Sample 6 | 0.716 |

Table 5 demonstrates that all of Test Samples 1 to 6 have a proliferation promoting effect on the Bifidobacterium.

### Test Example 6

Ten NC/Nga male mice (4-week old) were obtained from Japan SLC, and housed in a room at a temperature of 23±3° C. and a humidity of 55±15%, with a 12 hr-12 hr light-dark cycle (light on 7:00 to 19:00). The mice (5 per cage) were preliminarily fed a standard diet (Rabo MR Stock, Nihon Nosan Kogyo K.K.) for 7 days, and then divided into two groups (5 per group) to carry out the following test:

The mice of both groups were fed ad libitum. The test group was fed a diet prepared by adding 0.144 wt. % of freeze-dried product of crushed mixture of broccoli (44 wt. %) and cabbage (56 wt. %) to a MF powder diet (ORIENTAL YEAST K.K.) followed by blending in a mixer. The control group was fed a diet prepared by adding 0.144 wt. % of α corn starch (ORIENTAL YEAST K.K.) to the MF powder diet.

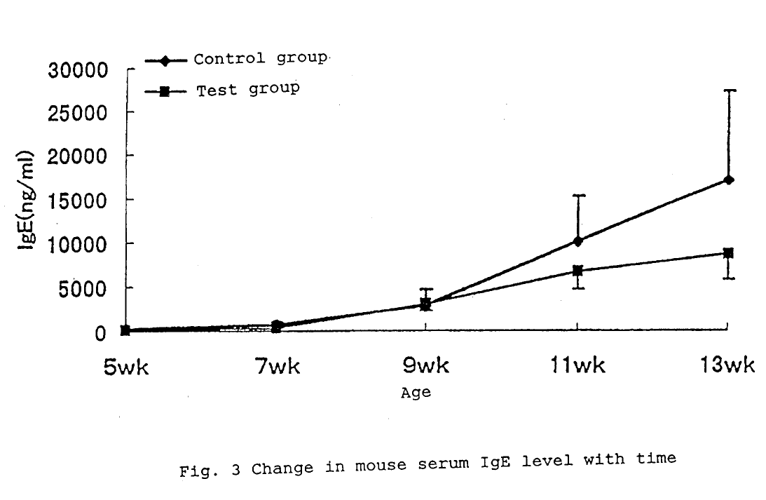

The serum IgE level of blood collected from the orbit of the mice of each group was measured using a mouse IgE kit “YAMASA” EIA (Lot. 702). The results are shown in FIG. 3.

As is apparent from FIG. 3, the serum IgE level increase in the test group was inhibited, as compared with the control group.

### Test Example 7

Test on Hepatocyte Cholesterol Synthesis Inhibitory Activity

(1) Composition and Method of Preparation of Test Sample

Juices obtained from the plant samples (1000 g each) shown in Table 6 were filtered through a gauze filter, and the resulting filtrates were centrifugally filtered at 10000 G. The supernatants obtained by the centrifugal filtration were freeze-dried to obtain the test samples shown in Table 7.

TABLE 6
Composition of plant sample (%)

| | Plant sample | | | | | | | |
|---|---|---|---|---|---|---|---|---|
| | T-1 | T-2 | T-3 | T-4 | T-5 | T-6 | T-7 | T-8 |
| Broccoli | 100 | — | — | — | — | — | 75 | 75 |
| Shepherd's | — | 100 | — | — | — | — | — | — |
| purse | | | | | | | | |
| Woad | — | — | 100 | — | — | — | — | — |
| Chinese | — | — | — | 100 | — | — | — | — |
| cabbage | | | | | | | | |
| Cabbage | — | — | — | — | 100 | — | 25 | — |
| Japanese | — | — | — | — | — | 100 | — | 25 |
| Radish leaves | | | | | | | | |
| Total | 100 | 100 | 100 | 100 | 100 | 100 | 100 | 100 |

TABLE 7
Test sample

| | Plant sample | | | | | | | |
|---|---|---|---|---|---|---|---|---|
| | T-1 | T-2 | T-3 | T-4 | T-5 | T-6 | T-7 | T-8 |
| | Test sample | | | | | | | |
| | S-1 | S-2 | S-3 | S-4 | S-5 | S-6 | S-7 | S-8 |
| Weight of test | 65 | 42 | 54 | 41 | 54 | 36 | 62 | 58 |
| sample after | | | | | | | | |
| freeze-drying | | | | | | | | |
| (g) | | | | | | | | |

(2) Test Method

The Test Samples S-1 to S-8 shown in Table 7 were used as test materials, and tablets containing 5.6% of pravastatin sodium (tradename: Mevalotin) were used as a positive control material. These materials were ground in a mortar and used.

HepG2 cells (2×10⁵ cells) cultured in DMEM containing 10% FCS were inoculated into 6-well plates and incubated for 3 days (substantially confluent). After removing the culture media, the test materials S-1 to S-8 and positive control material, all diluted with DMEM and mixed with ¹⁴C-acetic acid, were placed individually in the 6-well plates, followed by 2-hour incubation. Then, the culture media were removed, and the cells were washed with PBS, dissolved in KOH and saponified. The saponified products were subjected to ether extraction and exsiccated.

The exsiccated extracts were dissolved in acetone-ethanol, and 0.5% digitonin was added to each of the solutions. Thereafter, the radioactivity level of the resulting precipitates was measured using a scintillation counter.

(3) Test Result

Table 8 shows the test results, i.e., concentrations of test materials and positive control material which cause 50% inhibition of cholesterol synthesis in HepG2 cells.

TABLE 8
Concentration for 50% inhibition of cholesterol
synthesis

| | | Titer ratio | |
|---|---|---|---|
| | | relative to | Corrected titer |
| | Concentration | positive control | based on yield |
| Test sample | (%) | material | (Reference: S-7) |
| S-1 | 0.49 | 1/245 | 1/234 |
| S-2 | 0.37 | 1/185 | 1/273 |
| S-3 | 0.46 | 1/230 | 1/264 |
| S-4 | 0.40 | 1/200 | 1/302 |
| S-5 | 1.09 | 1/545 | 1/626 |
| S-6 | 0.78 | 1/390 | 1/672 |
| S-7 | 0.42 | 1/210 | 1/210 |
| S-8 | 0.40 | 1/200 | 1/213 |
| Positive | 0.002 | 1/1 | 1/1 |
| control | | | |
| material | | | |
| (Mevalotin) | | | |

In Table 8, “titer ratio relative to positive control” indicates a value obtained by dividing 0.002% (the concentration of the positive control (Mevalotin) for 50% inhibition of cholesterol synthesis) by the concentration of each test material for 50% inhibition of cholesterol synthesis. Thus, the value indicates the titer ratio of each test material to an equal weight of the positive control material.

“Corrected titer based on yield (reference: S-7)” in Table 8 is a value obtained by correcting the titer ratio relative to positive control based on the yield of each test material found from Table 7, using Test Sample S-7 as a reference. This value is presented so that the cholesterol synthesis inhibitory activities of the original plant samples for preparing the test materials can be compared with one another.

Theoretically, therefore, when doses of Test Sample S-7 and the positive control material (Mevalotin) which show an equivalent serum cholesterol lowering action are found from the results of a clinical test of administration of Test Sample S-7 and the positive control material to a hypercholesterolemia group, a suitable dose of each plant sample can be calculated based on the corrected titer.

In Test Example 7, the cholesterol synthesis inhibitory activity was found only in the water soluble fraction of each test sample. The ethanol fraction, acetone fraction and ethyl acetate fraction of each test sample were also tested for cholesterol synthesis inhibitory activity, but were found to have no such activity.

### Test Example 8

Human Clinical Test

Among the test samples found to have a hepatocyte cholesterol synthesis inhibitory activity in Test Example 7, Plant Samples T-1, T-5 and T-7 and Test Sample S-7 were used in a human clinical test on groups of hypercholesterolemia subjects, to determine the presence or absence of a clinical effect, and the correlation between the clinical effect and the cholesterol synthesis inhibitory activity.

(1) Content of Clinical Test

(1-1) Eighty hypercholesterolemia subjects were divided into 4 groups. Each subject was given two cans daily of the test samples (canned drinks) shown in Table 9, for 4 weeks, to evaluate the improving effect on blood indices such as serum cholesterol level.

(1-2) Blood was taken before and after the 4-week test period (Week 0 and Week 4).

(1-3) During the test period, the subjects were allowed to continue their usual diet and activity, without dietary restrictions or programmed exercises.

(2) Clinical Test Subjects

The clinical test was performed on hypercholesterolemia patients meeting the following criteria.

(2-1) Selection Criteria

i) Patients with a serum total cholesterol level of 240 (mg/dl) or higher

ii) Patients who understood the purpose and content of the test and consented to serve as subjects

(2-2) Exclusion Criteria

i) Patients with a complication of a hepatic disease, diabetes or like disease

ii) Patients given a therapeutic agent under treatment of hypercholesterolemia

iii) Patients regarded as unsuitable by the doctor for any other reasons

(3) Test Sample

Vegetable juices squeezed from Plant Samples T-1, T-5 and T-7 shown in Table 6 were filtered, and the filtrates were packed into cans (160 g per can), giving test samples. Further, another test sample was prepared by packing, per can, 160 g of a solution of 9.92 g of Test Sample S-7 (corresponding to 160 g of Plant Sample T-7) in purified water.

TABLE 9
Test sample for Text Example 8

| | | | Weight of | |
|---|---|---|---|---|
| Test | Content and its | | final | |
| sample | weight (g) | | product (g) | Daily dose |
| O-1 | T-1 | 60 | 160 | 2 cans (320 g) |
| O-2 | T-2 | 60 | 160 | 2 cans (320 g) |
| O-3 | T-7 | 60 | 160 | 2 cans (320 g) |
| O-4 | S-7 | 0.92 | 160 | 2 cans (320 g) |

(4) Test Result

Table 10 shows the change and rate of reduction before and after the clinical test in total cholesterol level in the four test groups. Table 11 shows the change and rate of reduction in LDL cholesterol level.

TABLE 10

| | Change in total cholesterol level | | |
|---|---|---|---|
| | | | Rate of change (%) |
| Subject | Week 0 | Week 4 | {Amount of change |
| group | (mg/dl) | (mg/dl) | (mg/dl)} |
| Group | 253.4 ± 26.9 | 226.5 ± 23.3** | ▴10.6 |
| O-1 | | | (26.9) |
| Group | 252.6 ± 23.1 | 243.8 ± 21.5* | ▴3.5 |
| O-2 | | | (8.8) |
| Group | 252.4 ± 27.9 | 223.3 ± 24.6** | ▴11.5 |
| O-3 | | | (29.1) |
| Group | 253.7 ± 26.3 | 223.5 ± 22.3** | ▴11.9 |
| O-4 | | | (30.2) |

In the table,
*indicates P < 0.05, and
**indicates P < 0.01.

TABLE 11

| | Change in LDL cholesterol level | | |
|---|---|---|---|
| | | | Rate of change (%) |
| Subject | Week 0 | Week 4 | {Amount of change |
| group | (mg/dl) | (mg/dl) | (mg/dl)} |
| Group | 171.4 ± 23.0 | 147.5 ± 21.5** | ▴13.9 |
| O-1 | | | (23.9) |
| Group | 168.2 ± 26.5 | 160.5 ± 23.3 | ▴4.6 |
| O-2 | | | (7.7) |
| Group | 170.7 ± 23.3 | 144.6 ± 21.7** | ▴15.3 |
| O-3 | | | (26.1) |
| Group | 171.0 ± 24.8 | 143.3 ± 21.1** | ▴16.2 |
| O-4 | | | (27.7) |

In the table,
**indicates P < 0.01.

Table 10 reveals that intake of the food of the present invention lowered the total cholesterol level in all of Groups O-1 to O-4.

In particular, the total cholesterol level in Group O-4 was lowered by 11.9% on average, by 4-week intake. As compared with the rate of reduction achieved by different doses of Mevalotin (the positive control material used in Test Example 1), the rate of reduction in Group O-4 was substantially equivalent to the rate of reduction achieved by 4-week administration of Mevalotin in a daily dose of 5 mg (11.1%). The prescribed daily dose of Mevalotin is 10 mg, which can lower the total cholesterol level by 18 to 20%.

The amount of change in cholesterol level shown in Tables 10 and 11 reveals that the main factor of the reduction in total cholesterol level is the reduction of LDL cholesterol.

Comparing the corrected titers shown in Table 8 and the results shown in Table 10, the rate of reduction in Group O-3 (T-7) was ▴11.5%, which is nearly equal to the rate of reduction in Group O-4 (S-7) of ▴11.9%. This substantially agrees with the fact that, in the case of Test Sample S-7, the ratio of the corrected titer to the original titer is 1:1.

The rate of reduction in Group O-1 (T-1) was ▴10.6% while the rate of reduction in Group O-4 (S-7) was ▴11.9%. The ratio of the two values (▴10.6%:▴11.9%) is 0.89:1, which is nearly equal to the corrected titer ratio of Test Sample S-1 to Test Sample S-7 (0.90:1). Further, the ratio of the rate of reduction in Group O-5 (T-5) (▴3.5%) to the rate of reduction in Group O-4 (S-7) (▴11.9%) is 0.29:1, which is nearly equal to the corrected titer ratio of Test Sample T-5 to Test Sample S-7 (0.34:1).

As to safety indices, no change was found in liver-related indices such as GOT, GPT, ALP, γ-GTP and LDH-5, or in cardiac muscle- or skeletal muscle-related indices such as CPK, LDH-1, LDH-2, LDH-3, LDH-4, creatine and creatinine, or in kidney-related indices such as BUN, creatinine and uric acid.

As to nutrition sufficiency indices, no change was found in serum total protein, albumin, immunoglobulin, choline esterase, serum amyrase or like indices, or in erythrocyte counts, hemoglobin level, platelet counts, hematocrit, leukocyte counts, leukocyte fractions (such as basophilic leukocytes, acidophilic leukocytes, lymphocytes and monocytes) or like indices.

As is apparent from the above, the human serum cholesterol lowering action of the food of the present invention is exhibited chiefly by the mechanism of hepatocyte cholesterol synthesis inhibitory activity. Namely, the food of the invention selectively reduces the low-density lipoprotein cholesterol (LDL-Chol) to thereby lower the serum total cholesterol, hence effective for improving lipid metabolism.

The serum cholesterol lowering action of the food of the invention was low as compared with Mevalotin, a medicine, but the food of the invention was completely free from side effects on the liver or cardiac muscle.

Therefore, the food of the invention is sufficiently significant for the purposes of keeping the good health of persons with slightly higher serum cholesterol level than normal level, since, according to the invention, human serum cholesterol can be lowered by taking a mixture of commonly eaten vegetables, which are free from the risk of unknown side effects usually entailed by novel medicines.

### Comparative Example 1

The following are the results of a test wherein rats were given a mixed vegetable juice containing suitable amounts of broccoli, cabbage, Japanese radish leaves, celery, lettuce, spinach, komatsuna, parsley and apple.

Ten SD-SPF rats (male, 4-week old) were divided into 2 groups. Group A (control group) was fed only a standard diet ad libitum, while Group B (test group) was fed a standard diet and the mixed vegetable juice ad libitum.

Generally, excessively high-fat diets such as fat-loaded diets are not employed during prophylactic or therapeutic treatment for lowering the cholesterol level, even when the treatment do not involve dietary restrictions. In the test examples of this invention, therefore, the rats were fed a standard diet, not a fat-loaded diet, to evaluate the serum cholesterol lowering action of the mixed vegetable juice.

Table 12 shows the total cholesterol level in each group before and 4 weeks after the start of the test.

TABLE 12

| | Change in total cholesterol level | | |
|---|---|---|---|
| | | | Rate of change (%) |
| Subject | Week 0 | Week 4 | {Amount of change |
| group | (mg/dl) | (mg/dl) | (mg/dl)} |
| Group | 111.8 | 74.2 | ▴ 33.6 |
| A | | | (37.6) |
| Group | 114.6 | 92.2 | ▴ 19.5 |
| B | | | (22.4) |

The total cholesterol level lowered not only in Group B given the mixed vegetable juice in addition to the standard diet, but also in Group A given only the standard diet. Rather, the amount and rate of change in Group A, which was fed the standard diet alone, were greater than those in Group B.

The above results demonstrate that the mixed vegetable juice containing broccoli, cabbage, Japanese radish leaves and other vegetables does not have an action of lowering the total cholesterol of rats.

The following are formulation examples of foods and medicines according to the present invention.

Formulation Example 1 Tablets

| | |
|---|---|
| Lactose | 80.0% |
| Gelatin | 10.0% |
| Freeze-dried broccoli | 1.0% |
| Freeze-dried cabbage | 1.0% |
| Fruit flavor | 0.5% |
| Water | 7.5% |

Formulation Example 2 Gum jelly

| | |
|---|---|
| Gum arabic | 18.0% |
| Galactooligosaccharide | 23.0% |
| Concentrated kale juice | 9.0% |
| Concentrated Broccoli juice | 9.0% |
| Starch syrup | 23.0% |
| Flavor | 0.5% |
| Pigment | 0.05% |
| Water | 17.45% |

Formulation Example 3 Beverage

| | |
|---|---|
| Apple juice | 20.0% |
| Broccoli puree | 20.0% |
| Cabbage juice | 10.0% |
| Concentrated lemon juice | 0.5% |
| Pigment | 0.05% |
| Flavor | 0.5% |
| Water | 48.95% |

Formulation Example 4 Granules

| | |
|---|---|
| Lactose | 90.0% |
| Talc | 0.5% |
| Freeze-dried cabbage | 5.0% |
| Fruit flavor | 0.5% |
| Water | 4.0% |

Formulation Example 5 Capsules

| | |
|---|---|
| Freeze-dried powder of | 10.0% |
| Bifidobacterium | |
| Freeze-dried Broccoli | 50.0% |
| Gelatin | 5.0% |
| Glycerin | 35.0% |

1. A food containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

2. A food according to claim 1, which contains at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

3. A food according to claim 1, which contains at least one member selected from the group consisting of broccoli, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), Chinese cabbage, hinona (Brassica campestris var. akana), sugukina (Brassica campestris var. neosuguki), turnip, rape, cabbage, brussels sprouts, Japanese radish and Japanese radish leaves.

4. A food according to claim 2, which has a Bifidobacterium proliferation promoting action.

5. A food according to claim 2, which has an allergy preventive action.

6. A food according to claim 3, which has a human serum cholesterol lowering action.

7. A medicine containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

8. A medicine according to claim 7, which contains at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

9. A medicine according to claim 7, which contains at least one member selected from the group consisting of broccoli, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), Chinese cabbage, hinona (Brassica campestris var. akana), sugukina (Brassica campestris var. neosuguki), turnip, rape, cabbage, brussels sprouts, Japanese radish and Japanese radish leaves.

10. A medicine according to claim 8, which is a preventive or therapeutic agent for an allergy.

11. A medicine according to claim 9, which is a preventive or therapeutic agent for human hypercholesterolemia.

12. A medicine according to claim 9, which is a preventive or therapeutic agent for human arteriosclerosis.

13. A cosmetic having an action of alleviating rough skin conditions, containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

14. A cosmetic according to claim 13, which contains at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

15. A method for preventing or treating a disease, comprising ingestion or administration of an effective amount of at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

16. A method for preventing or treating a disease according to claim 15, which comprises ingestion or administration of an effective amount of at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

17. A method for preventing or treating a disease according to claim 15, which comprises ingestion or administration of an effective amount of at least one member selected from the group consisting of broccoli, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), Chinese cabbage, hinona (Brassica campestris var. akana), sugukina (Brassica campestris var. neosuguki), turnip, rape, cabbage, brussels sprouts, Japanese radish and Japanese radish leaves.

18. A method for preventing or treating a disease according to claim 16, wherein the disease is an allergy.

19. A method for preventing or treating a disease according to claim 17, wherein the disease is human hypercholesterolemia.

20. A method according to claim 17, wherein the disease is human arteriosclerosis.

21. A method for proliferating a Bifidobacterium, comprising contacting with the Bifidobacterium an effective amount of at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

22. A method according to claim 21, which comprises contacting with the Bifidobacterium an effective amount of at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.

23. A method for alleviating rough skin conditions, comprising applying to the skin an effective amount of a cosmetic containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce, apple, Chinese cabbage, brussels sprouts, Japanese radish and Japanese radish leaves.

24. A method according to claim 23, which comprises applying to the skin an effective amount of a cosmetic containing at least one member selected from the group consisting of broccoli, cauliflower, kale, shepherd's purse, suzushiro (Arabis flagellosa Miq.), hatazao (Arabis glabra), tagarashi (Cardamine flexuosa), woad, oobatanetsukebana (Cardamine scutata), yamagarashi (Barbarea vulgaris R.Br. var. stricta Regel), watercress, takana (Brassica juncea Czern. et Coss. var. integrifolia Sinsk.), karashina (Brassica juncea Czern. et Coss.), Japanese horseradish, yuriwasabi (Wasabia tenuis), hinona (Brassica campestris var. akana), sugukina (Brassica campestris var. neosuguki), turnip, rape, cabbage, spinach, komatsuna (Brassica rapa L.), celery, parsley, lettuce and apple.
